(19) United States
(12) Patent Application Publication
(10) Pub. No.: US 2010/0099956 A1
(43) Pub. Date: Apr. 22, 2010

(54) Wireless Device and Method for Secure Auto Transfer of Medical Device Information to a Network of Wireless and Non-Wireless Devices

(75) Inventors: Stephen Richard Cole, Durham, NC (US); Lyle Glenn Bullock, Honolulu, HI (US); John Francis Petro, Raleigh, NC (US)

Correspondence Address: WARD AND SMITH, P.A., 1001 COLLEGE COURT, P.O. BOX 867, NEW BERN, NC, 28563-0867, US

(73) Assignee: CONFIDANT HAWAII, LLC, Honolulu, HI (US)

(21) Appl. No.: 12/578,125

(22) Filed: Oct. 13, 2009

Related U.S. Application Data
(60) Provisional application No. 61/104,907, filed on Oct. 13, 2008.

(51) Int. Cl. A61B 5/00

(52) U.S. Cl. 600/300

(57) ABSTRACT

A medical system includes a meter adapted for taking biometric measurements. A wireless communication module is associated with the meter for transmitting data representing the measurements. Collector devices are adapted to receive the data when a connection is available, process the data and transmit the data to a server. The server may be a HIPAA compliant server.

### CROSS REFERENCE TO RELATED APPLICATIONS

This application is related to and claims priority to Provisional Application Ser. No. 61/104,907, filed Oct. 13, 2008 entitled A Wireless Device and Method for Secure Auto Transfer of Medical Device Information to a Network of Wireless and Non-Wireless Devices, the disclosure of which is specifically incorporated by reference herein in its entirely.

### BACKGROUND OF THE INVENTION

1. Field of the Invention

This invention relates to a multipart system including a wireless module embedded in/on a medical device that is used for biometric collection of data. The wireless module automatically searches for, and wirelessly “transmits” data to at least one of many possible pre-assigned network elements. The data is then distributed to all the pre-assigned network elements. The invention also relates to a system of devices networked in such a way as to provide secure transfer and relay of data in order to make it visible to all authorized users. The system allows text messages and e-mails, etc. to be transmitted to other locations and parties, allowing intervention to occur, if necessary, based on data collected.

2. Discussion of Prior Art

People with a chronic disease (e.g., diabetes) or health problem (e.g., obesity) are often left alone to manage their disease on a daily basis. This has been traditionally a problem among adults, and self monitoring by adults of their own condition was typically employed as a solution or control of the condition. However, more recently there has been an alarming trend toward a high incidence of chronic disease in children (e.g., diabetes and asthma). This has lead to a desire by concerned parents and guardians to monitor their children throughout the day.

A major use of technology for personal management of a chronic disease is through the use of biometric measuring devices (e.g., a glucose meter, peak flow meter, blood pressure meter). These devices are used to monitor a chronic disease over time, and it is very valuable for a health care provider or parent to view a series of measurements over time, to determine the best course of treatment.

Today, there are a multitude of software and device interfaces available that can perform computerized data collection either through a PC, PDA, or a mobile phone. All such devices have one thing in common, i.e., they require various levels of user interaction, from plugging in of cables to initiating applications on the various devices.

There are in particular two identified situations in which an automatic transfer and observation of the measurement data would be very valuable. One such situation involves children who have to take measurements while at school. A second situation arises in nursing homes. In both of these cases there may be barriers to user activated data transfer to a computer or mobile device, either through school policy (e.g., no cell phones allowed and complexity), or in nursing homes (because of complexity, accuracy and overhead required).

Among many current prior art solutions, many companies provide data collection software packages. However, such packages all require some user interaction, often at a complicated level. Companies such as Johnson & Johnson, Abbott Diabetes Care, Agamatrix, and Roche have such products commercially available.

Some prior art systems such as one available from Mitsumi Corporation, include Bluetooth modules which are embedded in blood pressure meters, scales and at least one glucose meter. However, none of these systems are capable of automatically transferring data without user/patient interaction, and/or are not part of a system that can relay data across a network of devices.

In addition to the foregoing, many companies are currently exploring automatic transfer devices, but they are not implemented or implementable in a system, or in a manner which preserves battery life in the devices.

### SUMMARY OF THE INVENTION

In accordance with the invention, there is provided a new wireless solution integrated in a communications system or network.

Such a system includes a medical device for taking, or capable of taking, biometric measurements. The medical device such as a meter is adapted for taking biometric measurements from a user, and by the term “biometric measurements” is meant [fill in definition.] A wireless communication module is associated with the medical device or meter for conducting wireless transmission of data representing biometric measurements taken by or with the medical device.

The wireless communication module is optionally programmed for connecting in a wireless manner to a network. Further, the communication module may be configured to connect when signaling is established after a period of time of no availability, for example, by periodic polling of network signal availability. Optionally, the communication module, e.g., a wireless master module, is programmed for transmitting the data, for example, when a signaling is established, without user interaction.

In another aspect, a method of obtaining and transmitting data representative of biometric measurements from a user is provided. Biometric measurements are obtained through a medical device and wirelessly transmitted to a remote server. The data is then transmitted in a wireless manner to a network.

Other aspects of the method are implemented as described briefly with reference to the system previously described.

In accordance with the invention, the following is provided:
1. Wireless transfer of data is provided from a medical device, for example, to a device such as a computer or mobile phone without requiring user actions or interaction;
2. Wireless transfer of data may be provided from multiple medical devices of multiple users, for example, to the same computer or a mobile phone without requiring user actions or interaction;
3. Because of intermitting polling of network signal (as opposed to continuous “on”) a prolonged battery life (>2 months) is achievable;
4. The system can be configured as a star network; and,
5. There is an ability to reconnect to a network, even if all signaling between the devices and the network is lost for some time.

The automated transfer of data to a communication system also allows for the following:
1. Biometric measurement data may be entered into the system in a timely way without user interaction;
2. Parents and guardians of children having a chronic disease may monitor their children's measurements and compliance;
3. In addition, measurement data will trigger the start up of an application that can:
   a. Provide feedback to appropriate users;
   b. Institute a question and answer (“Q&A”) session that can supply the system with additional information such as medication usage, and health symptom information;
   c. Bypass an application start up by the user, which can be very cumbersome and hard to find on some mobile phones; and,
   d. Data will eventually transfer, even though no receiving device is immediately available; and,
4. Increased staff productivity and data accuracy in nursing homes is achieved.

### BRIEF DESCRIPTION OF THE DRAWINGS

Having briefly described the invention in summary form, the same will become better understood from the following detailed description made with reference to the appended drawings, wherein:

FIG. 1 is a system block diagram illustrating transfer and distribution of measured data, and response thereto when appropriate;

FIG. 2 is a diagram illustrating how data is transferred in the system, depending on the type of “collector” receiving the data;

FIG. 3 is a flow diagram illustrating how a “wireless master module” searches for a connection for transmission of data;

FIG. 4 is a block network diagram illustrating communication between a wireless master model through various networks and devices to a medical device or meter having a wireless master module associated therewith;

FIG. 5 is a block diagram illustrating “master module” logic; and,

FIG. 6 is a flow diagram illustrating operation of the master module.

### DETAILED DESCRIPTION OF THE INVENTION

The extent of probable use of the invention described herein is predicted to include millions of users based on the chronic illnesses estimates of diabetes and asthma alone.

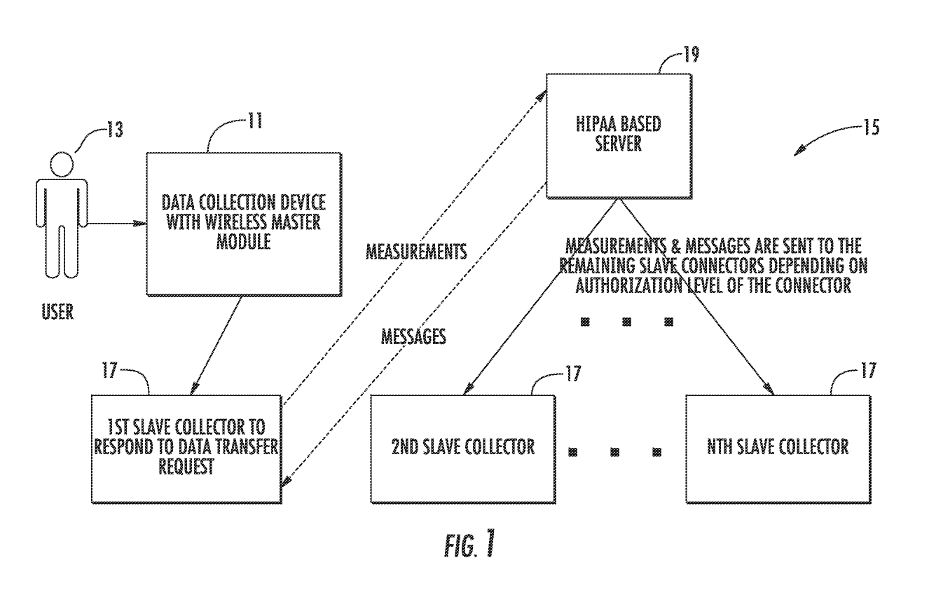

As shown in FIG. 1, a low power wireless module can be embedded or coupled with a biometric measurement device 13 taking measurements from a user 13, and programmed to automatically transfer biometric data to any one in a network of other wireless devices. A network system 15 of devices that can perform various levels of communication and data transfer based on authorizations and permissions is provided. Exemplary system 15 parts are labeled in the block diagram and element descriptions of FIG. 1. The diagram illustrates the transfer and distribution of measured data, and responses as applicable.

The system 15 of FIG. 1 may include a series of collectors 17 which receive data from the biometric measurement device 11. A first type of collector 17 is a “secure relay collector.” A secure relay collector may be a communication server which may resident in a non secure environment. It is configured to receive encrypted data from the device 13 and relay the encrypted data to, for example, a server 19 in a HIPAA compliant environment, i.e., a HIPAA based server. The secure collector 17 may be a standard wireless enabled device (PC, PDA, Cell Phone etc.) as will readily be apparent to those of ordinary skill.

Other functions of a secure collector is that it is capable of connecting to the internet, and can connect automatically, due to programming, when it receives data from the meter or biometric measuring device 11. The secure relay collector is programmed to communicate with multiple meters 11, each with wireless master communication modules, and with multiple simultaneous connections. The secure collector is secured so no user can access the data received and continued therein. In addition, it operates as a wireless slave normally, but can act as a wireless master device when required.

A second type of collector 17 is known as a “guardian collector.” The guardian collector is a communications server with a number of specific functions and properties. It may be resident in a non-secure environment. In addition, it may be programmed to receive encrypted data from the meter 11 and relay the encrypted data to, for example, a HIPAA based server 19. The guardian collector may be a standard wireless enabled device (e.g., PC, PDA, Cell Phone etc.), and may be capable of connecting to the internet. It is also programmed to connect automatically when it receives data. The guardian collector is also programmed so that it can communicate with multiple meters 11 having wireless master modules, and having multiple simultaneous connections capabilities. The guardian collector provides limited user access to the data and messages, depending on authorization level. It can also function as a wireless slave under normal operation, but can operate as a wireless master when required.

A third alternative to the collector 17 is a “full feature collector.” The full feature collector may be a communications server capable of being resident in a non-secure environment. It may receive encrypted data from the meter 11 and relay the encrypted data to, for example, the HIPAA based server 19, or other type of “central” server or device. The full feature collector is a standard wireless enabled device (for example, PC, PDA, Cell Phone etc.). It can also connect to the internet, and connects automatically when it receives data.

A further feature of the full feature collector is that it can communicate with multiple meters 11, having wireless master modules, and having multiple simultaneous connections. It provides full user access to the data and messages. It is a wireless slave under normal operation, but can be a wireless master when required.

The central server 19, is typically a HIPAA compliant based server, and, for example, resides in a HIPAA compliant service center. It preferably has secure login/password access, with varying privileges depending on degree of authorization. In operation, users may view data, or portions of data, depending on authorization level, and users can configure accounts if authorized. The server 19 may contain a list of configured network, in one embodiment Bluetooth standard identification indicia, i.e., “BT IDs” for each measurement device or meter 11. In addition, after receiving data for a particular measurement device 11 having a wireless master module, it transmits the data to all slave collectors 17 from the wireless master module. The data collection device 11 operates to collect measurements from the user 13. The data collection device 11 contains or is attached to the wireless master module (not numbered), which is functionally a part thereof. The data collection device 11 (or meter) may automatically transmit data to the wireless master module. If data is not automatically transmitted then the data collection device 11 transmits the data in response to a poll for data from the wireless master module. The wireless master module is located on or is attached to the measurement device 11. It is automatically activated when it receives a measurement. It may also configured or programmed by a wireless master device, such as a collector 17, that may revert to slave mode for future communications.

As a general rule, the wireless master module operates in slave mode until it is configured. Upon being configured it operates as a master of a network of slave wireless devices or collectors 17. Once the wireless master module is configured, it will only communicate with its slave devices (i.e., collectors 17), and only needs to transmit data to one slave device, i.e., collector 17.

The wireless master module can be configured to search a slave list based on priorities to conserve power, and it goes into low power mode after transfer of data to one slave device or collector 17. The wireless master module supports data encryption, PIN/ID authentication, and supports three different peripheral modules. The peripheral modules may include a processor module that can poll for measurement data from the measurement device 11 when the measurement device 11 does not automatically send its data to the wireless master module. Another peripheral module may be a low profile power module using coin cell battery technology. It may also include a low profile power module using rechargeable battery technology, and has a programming interface.

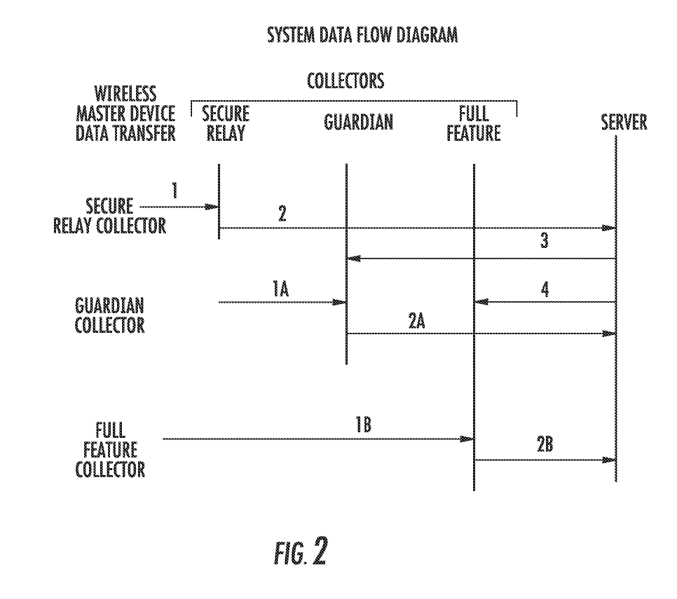

FIG. 2 illustrates how data may be transferred depending on the type of collector 17 that receives the data. The 3 possibilities are shown as 1, 1A and 1B.

The biometric measurement device 11 first contact is with a secure relay collector (SRC) (e.g., a school computer). It transfers its data through the wireless master module to the SRC. The SRC then relays the data 2 to, for example, the HIPAA based server 19. The only communication back to the SRC and to the biometric measurement device 11 is an acknowledgement of receipt of data (ACK). The HIPAA based server 19 then sends, 3 and 4, the appropriate information and application activation to the guardian collectors and to the full feature collectors.

At 1A, the biometric measurement device 11 first contact is with a guardian collector (e.g., parent's mobile phone). It transfers its data to the guardian collector that then relays the data 2A to the HIPAA based server 19. The HIPAA based server 19 then sends, 3 and 4, the appropriate information and application activation to the other guardian collector and to the full feature collector. There is no communication to the SRC if one is in the network.

At 1B, the biometric measurement device 11 first contact is with a full feature collector (e.g., patient's mobile phone). The device 11 transfers its data to the full feature collector. The full feature collector then relays 2B the data to the HIPAA based server 19. The HIPAA based server 19 then sends, 3 and 4, the appropriate information and application activation to the guardian collector and to the other full feature collector. There is no communication to the SRC if one is in the network.

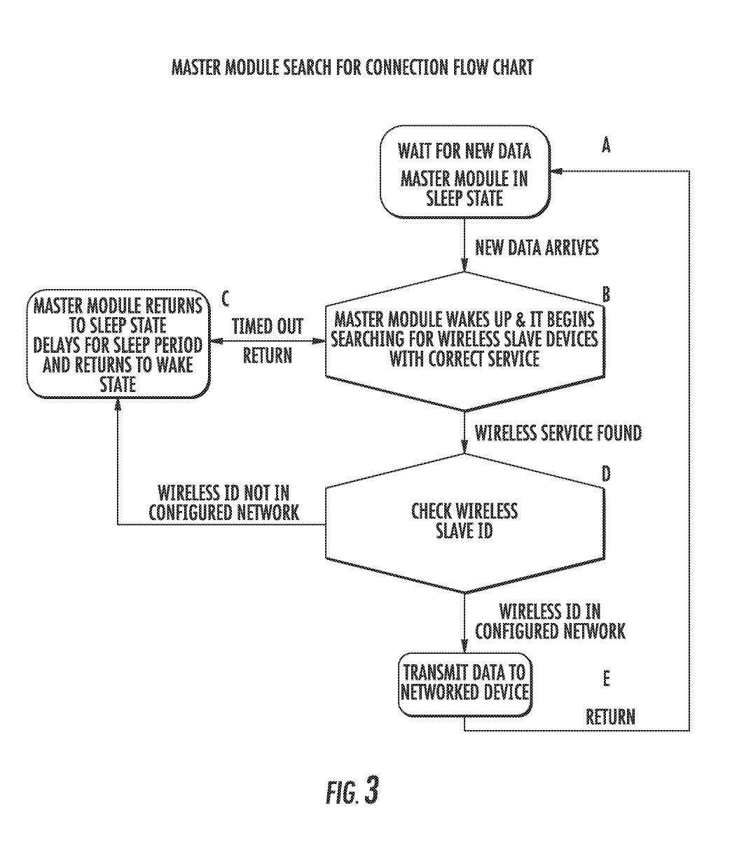

The following discussion provides an explanation of the flowchart of FIG. 3:

At step A, the wireless master module is in a sleep state while it waits for data. By this is meant that a radio chip thereon that consumes a majority of the power is not active. Most radio chips support a sleep state with various “wake up” triggers and in any case can always be awakened by an inexpensive low power processor (e.g., PIC processor) on the wireless module that can wake the radio chip via a reset line. If there is no sleep state then a standard shut down circuit (e.g., see application notes for the LTC1981 chip made commercially available by Linear Technologies) can be used, and the reset line can again be used to start the radio chip. The low powered processor can also be used to poll the medical device or meter 11 when the wireless master module is not integrated within the medical device 11, but is only attached to a physical port.

At step B, when the wireless master module is active and searching, it may not immediately find an acceptable service. It can be programmed over a wireless interface with a search time out period, and a wake again time out period, in order to conserve power when no slave device, i.e., collector 17 is readily available. This is a standard feature on many wireless radio chips (e.g., Bluecore4 chip commercially available from Cambridge Silicon Radio). When the wireless master module is capable of connecting to any one of a number of slave devices or collector 17, but only able to search for one device or collector 17 at a time, the wireless master module can be programmed to prioritize the search order using different sets of programmed criteria.

At step C, when the wireless master module does not find a wireless ID with the correct service, it will time out and move to step C where it sleeps for a period of time before returning for a new search. Again, this sleep period can usually be programmed over its wireless interface, but if not possible, it may have to be hard coded on the module or programmed over the air to a secondary processor as mentioned with reference to step A above.

At step D, if a wireless device, i.e., a collector 17, is found with the correct service, then the wireless master module checks its ID to see if it is on its network. If not, then it returns to step C. If it is on the network, then it begins to exchange information with the wireless slave device, i.e., slave collector 17. This exchange can also include, for example, a PIN change. The connection can be held until the slave collector 17 successfully communicates to, e.g., the HIPAA based server 19.

At step E, the data is successfully transmitted to, e.g., the HIPAA based server 19, where it is then relayed to all appropriate network devices depending on their authorization level.

The network 15 must be initially configured to implement the invention. More specifically, the configuration of a network with a wireless master device or module and multiple slaves such as collector 17 is described in many conventional and known wireless standards. For example, the Bluetooth wireless standard even allows for slaves to belong to multiple networks. The unique part of this invention is the system element that includes the server, such as a HIPAA based server 19, as a centralized communication point for all of the network elements. The value of this central point has already been demonstrated in the discussion of system data flow of FIG. 2. However, there is also value in using, e.g., the HIPAA based server 19 as an additional tool for network configuration. While the description is provided with reference to a HIPAA based server 19, it will readily be apparent that it can be implemented with other types of servers having similar functionality. Thus, by the term HIPAA based server is intended to mean HIPAA compliant servers as well as other non-HIPAA compliant servers having like or similar functionality.

For the configuration process, there is initially described two examples of slave collectors, i.e., mobile phones (these devices are usually assigned to the patient, parents, guardians) and personal computers (this could be a Home PC, school PC or a PC in a Doctor's office).

In the case of mobile phones, the phone numbers and authorization levels may be entered on the HIPAA based server 19 at the time the patient or user subscribes to the service. The HIPAA based server 19 can then send an application implementing aspects of the invention to the mobile phone. By the term “mobile phone” is intended conventional mobile phones, or like wireless devices having send and receive capabilities, such as MP3 players, and including devices like those available from Apple Inc. under the names iPod and PDA devices with wireless capabilities.

For a personal computer, requirements include an internet connection and compatible wireless technology. The typical application is as a relay server but this method work for all PCs. An application may be loaded on the PC. The application supports multiple clients, i.e., meters 11 comprised of multiple medical devices. Once an application is loaded on the PC, it may have an add user button for the application. The application presents a screen for entry of a user name and/or password. The PC then contacts, e.g., the HIPAA based server 19 to obtain a wireless ID of the wireless master module of the meter 11. The PC is then able to pair as a slave to the wireless master module 11. PIN codes could also be required for added security.

The wireless master module configuration can be configured by any one of the slave devices or collector 17, but the most practical device to use is one of the full feature collectors 17. The most efficient way for the wireless master module to be configured is to reverse roles until it is configured. More specifically, the wireless master module operates in slave mode and the slave device, i.e., full feature collector, acts as a wireless master device while it is configuring the wireless master module. The reason for this operation is it is easier because the slave collectors 17 have more capability and knowledge than the wireless master module before it is configured. The major part of the configuration is identifying the wireless master module network. There are multiple engineering ways to do this as well known to those of ordinary skill. One way is to expose the wireless master module to each of the slave collectors 17. Configuration information may include medical device information, time out periods, storage requirements and any other features and functions that could be user specific.

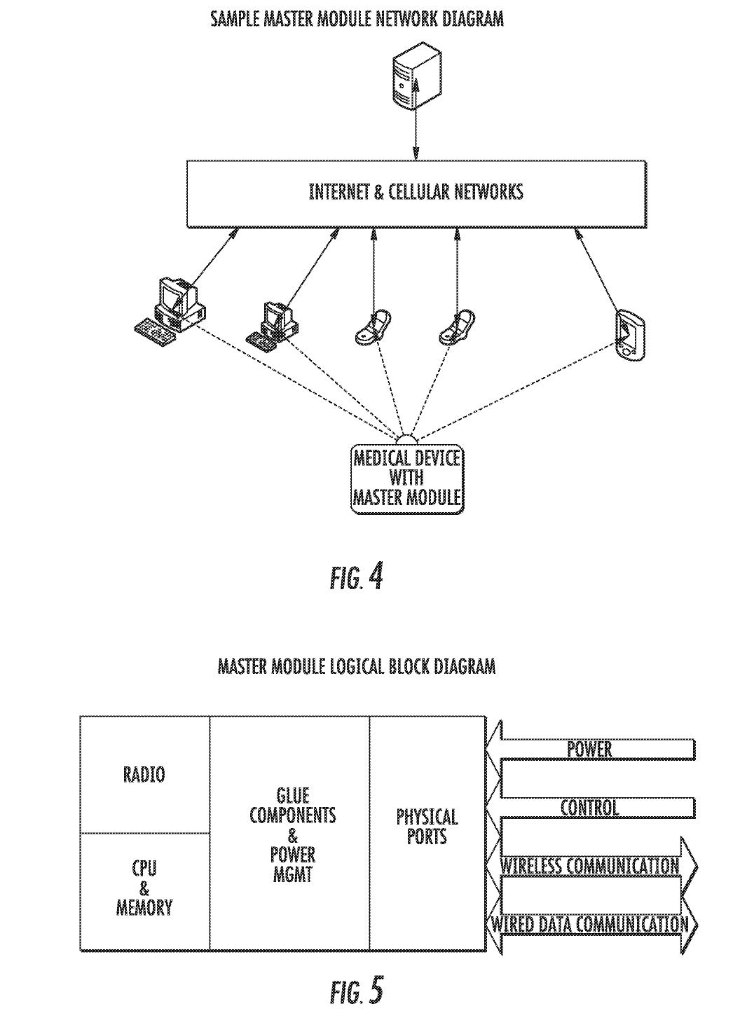

FIG. 4 illustrates a meter with a wireless master module 11 that can wirelessly communicate with any one of the devices 17 and 19 in its network, and that can then send the data to the central server 19 residing at a HIPAA compliant site. Each of the network devices 11, 17 and 19 is capable of supporting multiple wireless master modules, even if they belong to different users.

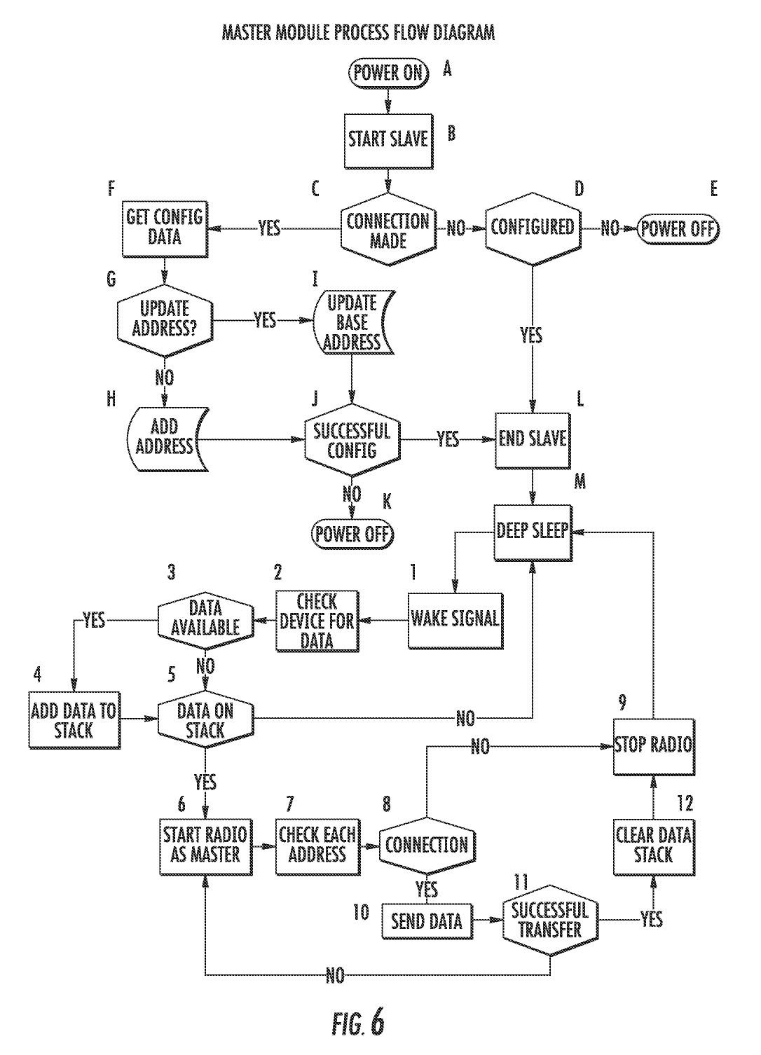

The wireless master module is shown as a device 31 in FIG. 5. It includes a radio, which is shown as the wireless radio block, and includes memory, an antenna, a radio chip set, a power interface and an oscillator. The device also includes a CPU and memory. A separate CPU and memory may not be needed in a fully integrated case for device 31, when the radio chip set also provides a CPU. However, it may be needed when the wireless master module is used as an attachment to the meter 11. The device 31 may then have its own oscillator and power interface.

A third block is shown as “glue components.” Glue components are the usual set of electronics needed to ensure a design functions properly. Glue components include elements such as resistors, capacitors, ESD protection, buffers, port drivers, logic, data converters, etc., as well known to those of ordinary skill. A power management module is also part of the same block as the glue components. This module is one physical area where the wireless master module differs from other wireless modules that exist today. The wireless master module will have power storage through the use of a “super capacitor” (a.k.a., ultra capacitor), well known to those of ordinary skill, along with the voltage regulation required for support. Such super capacitors have been used for a variety of applications since the mid 1990's, including as backup power storage for PCs. They are also currently being considered as battery replacements in hybrid cars.

In regard to FIG. 5, it is noted that one of the drawbacks of including a radio module on a device, is that radios often require high current to operate. Many medical devices are designed to operate on lithium coin cell batteries that have very low current output capability. For example, some coin cells batteries can only supply 0.2 mA of current while a standard Bluetooth radio may require over 50 mA to operate. The super capacitor addresses this discrepancy. More specifically, it can supply a high current after charging, and can be charged slowly by a standard lithium coin cell. This type of circuitry is perfect for many medical devices where a few measurements are taken over time each day. As such, high current requirements are met during activity, and long inactive periods are used for recharging from a lithium coin cell. Voltage regulation can allow for a large variation of input voltage to make the module compatible with existing electronics, without requiring the medical device manufacturer to change the power design on the medical device.

With respect to the power input shown, a standard power input can accept voltages having a wide range. A typical design will accept 2.2 V (Min) to 16 V (Max). However, both the Min and Max can be extended with added cost as well known to those of ordinary skill.

The control input is a standard set of typical module controls, that include, but is not limited, to a programming interface and a module reset. The wireless communication is the radio interface for data communications. The wired data communications is the physical interface/connections for data communication between the medical device 11 and the wireless master module.

Described hereinafter is a possible implementation of an illustrative implementation of Bluetooth wireless technology, which is a technology that supports all of the wireless requirements mentioned. The Bluecore4 module made by commercially available Cambridge Silicon Radio (CSR) is a programmable Bluetooth radio that can be programmed to support this invention. The system developed by a client/server system, specifically with a HIPAA based server 19 that supports this invention.

In FIG. 6, when the Bluetooth device, i.e., BT Device is powered on at step A, it starts in a slave mode at step B ready to accept connections from other devices. It stays in this mode typically for 30 seconds, waiting for a connection at step C. If no connection is made, it checks its configuration at step D to see if any slaves, i.e., collectors 17 have been configured. If there are no slaves or collectors 17 configured, then the device powers down completely at step E. If configuration has occurred, it will power down the radio at step L and go into a sleep mode at step M pending data and radio functions.

If a connection is made at step C, the data passed will indicate if this is an update to the base address or a new slave at step F. If the configuration information indicates an update to the base unit at step G, then the base address will be updated at step I. Otherwise, the address will be added to the list of additional authorized slaves at step H. If there is a successful transaction at step J, the device will shutdown the radio at step L and enter the deep sleep state at step M. If the configuration fails the device powers off at step K and allows the user to restart the process.

At step 1, resulting from a trigger which comes either from an internal timer or an attached/integrated device, the BT device will wake and begin the data/radio process. The device will check to see if a medical device is attached or integrated, and if it finds a device that it recognizes, it will query for data at step 2. If there is data available at step 3, it will add that data to the stack at step 4. In either case, the device will check the data stack to see if there is data pending transmission at step 5. If there data pending transmission it will start an instance of the radio in master mode at step 6. Otherwise, the BT device will once again enter deep sleep at step M.

When the device is operating in wireless master mode at step 6, it attempts a connection with each address in its list starting with the base address at step 7. If there is no connection at step 8, the BT device will shutdown the radio at step 9 and go into the deep sleep at step M until the trigger fires again, typically after approximately 90 seconds.

If there is a connection at step 8, the BT device will verify the connection request based on device address and a secret key/identifier. If successfully connected, the data will be transmitted to the connected radio at step 10. Upon successful transfer of the data to the requester at step 11, the data will be removed from the data stack at step 12, the radio shutdown at step 9 and the BT Device enters a deep sleep state at step M. If the transfer was not successful, the connection will be reset and another cycle will be started with restarting the radio at step 6.

With respect to device data stacks, the BT device will typically use the User PSKeys 0 to 49. Configuration data will start at 0 and work up as needed (note these can only be set during manufacturing or secure DFU). Data stack will be at 25 to 49. In addition, Authorized Addresses will be stored in PSKEY_LINK_KEY_ADDR0 to 16.

It is important to appreciate that the system can be configured to accomplish multiple communication functions. More specifically, text messages can be transmitted between network elements based on data analysis at the server 19. Messages can be sent to authorized third parties, either directly from the collector 17, the server 19, and in some cases appropriately configured directly to an interested third party such as a parent, guardian or other party responsible for the user taking biometric measurements. The third party may also receive, as appropriate, messages from collectors 17 and/or server 19, allowing intervention in the case of highly abnormal measured biometric data.

Having thus generally described the invention, the same will become better understood from the appended claims in which it is set froth in a non-limiting manner.

What is claimed is:

1. A medical device for taking biometric measurements, comprising:
a medical meter adapted for taking biometric measurements from a user; and,
a wireless communication module associated with the medical meter for wireless transmission of data representing biometric measurements taken by the medical device.

2. The medical device according to claim 1, wherein said wireless communication module is programmed for connecting wirelessly to a network.

3. The medical device according to claim 2, wherein said wireless communication module is programmed for connecting to said network when signaling is established after a period of time of no signal availability.

4. The medical device according to claim 1, wherein said wireless communication module is programmed for uploading and storing data representing biometric measurements until a wireless network signal becomes available, and for transmitting the data when the network signal becomes available.

5. The medical device according to claim 1, wherein said wireless communication module is programmed for transmitting said data automatically without user interaction.

6. The medical device according to claim 1, wherein the wireless communication module is programmed for transmitting data representing biometric measurements to network elements in a system, or directly to, or from network elements to a device associated with an interested third party.

7. A method of obtaining and transmitting data representative of biometric measurements from a user, comprising:
obtaining biometric measurements from a user through the use of a medical meter;
uploading the biometric measurements as data representative of said measurements into a wireless communication module associated with the medical meter; and,
wirelessly transmitting said data to a network.

8. The method of claim 7, further comprising searching for a wireless network with said wireless communication module when a network is not available, and connecting and transmitting said data to a network when one becomes available.

9. The method of claim 8, wherein said searching for a network when one is not available is conducted intermittently.

10. The method of claim 7, further comprising transmitting messages to a plurality of network elements or directly to, or from network elements to a device associated with an interested third party.

11. A system for collecting biometric measurements, comprising:
a medical meter adapted for taking biometric measurements from a user, having a wireless communication module associated with the medical meter for wireless transmission of data representing biometric measurements taken by the medical meter;
at least one collector device for receiving biometric measurements from said medical meter, and for transmitting said biometric measurements to a central server for processing, when a communications link to said server is available, and,
a central server configured for receiving biometric measurements from said at least one collector device, and for processing said measurements.

12. The system of claim 11, comprising at least one network connection for receiving said biometric data wirelessly, and said at least one network connection being adapted for receiving data from a plurality of medical meters when each one of said plurality is in signaling range.

13. The system of claim 11, wherein at least one collector comprises at least one of a plurality of network elements configured for transmitting a signal indicative that data has been received from a specific medical meter to a third party having a device associated with the third party and affiliated with said specific medical meter.

14. The system of claim 13, wherein the signal is a wireless signal transmitted to a wireless device of said third party for receipt of said signal by said wireless device of said third party.

15. The system of claim 11, wherein said at least one collector is a personal computer configured for receiving wireless signals and for sending data received wirelessly from said medical meter to other network elements.

16. The system of claim 11, wherein said central server is a HIPAA complaint server.

17. The system of claim 11, wherein said medical meter is configured for storing data representing the biometric measures when a connection to a collector is not available, periodically polling for a connection, and transmitting the data when a connection is available.

18. The system of claim 11, wherein said collector is configured for storing received data representing the biometric measures when a connection to the central server is not available, periodically polling for a connector and transmitting the data when a connection is available.

19. The system of claim 11, wherein said wireless communication modules is integrated with the medical meter.

20. The system of claim 11, wherein said wireless communication module is separate from and connectable to the medical meter.
